(19) United States
(12) Patent Application Publication
(10) Pub. No.: US 2024/0206523 A1
(43) Pub. Date: Jun. 27, 2024

(54) Compound of reaction of alkaloid and phenol, atomization liquid, cartomizer and electronic atomizer

(71) Applicant: Qi ZHANG, Shenzhen, Guangdong (CN)

(72) Inventor: Ting FENG, Suzhou, Jiangsu (CN)

(21) Appl. No.: 18/570,797

(22) PCT Filed: Jul. 5, 2021
(86) PCT No.: PCT/CN2021/104444

§ 371 Date: Dec. 15, 2023

(30) Foreign Application Priority Data
May 21, 2021 (CN) 202110558047.X

(51) Int. Cl. A24B 15/167; A24F 40/10; C07D 211/94; C07D 401/04

(52) U.S. Cl. CPC A24B 15/167; A24F 40/10; C07D 211/94; C07D 401/04

(57) ABSTRACT

Disclosed are a reaction compound of alkaloid and phenol, an e-liquid, an atomizing cartridge and an electronic atomizer. A reactant alkaloid and a reactant phenol are added into a reaction vessel, and heated and stirred continuously at 40-300° C. for 0.1-6000 minutes to obtain the reaction compound of alkaloid and phenol. In this way, the reaction compound of alkaloid and phenol, the e-liquid and the atomizing cartridge better the original choking irritation of the alkaloid in the electronic atomizer, which is beneficial for the mouth inhalation, does not cause troubles such as “acidosis”, “acid excess” and “sour smell”, and can provide a faster, more pleasant and satisfying suction experience.

### FIELD OF THE INVENTION

The present invention relates to the field of organic chemical synthesis and electronic atomization, in particular to a reaction compound of alkaloid and phenol, an e-liquid, an atomizing cartridge and an electronic atomizer.

### DESCRIPTION OF THE RELATED ART

Electronic atomizers, also known as e-cigarettes, are effective tools to help smokers quit smoking. Electronic atomizers are mainly divided into vapor electronic atomizer (Vapor) that consumes liquid atomizing cartridges and heat-not-burn (HNB) electronic atomizer that consumes solid atomizing cartridges. Compared with traditional tobacco, these two types of e-cigarettes can efficiently transmit nicotine, as well as greatly reducing the inhalation of tar, carbon monoxide, nitric oxide and other harmful substances into human body, so they have been used by hundreds of millions of users around the world as “harm reduction products”.

However, research on the hazards of e-cigarettes is not thorough. For example, the nicotine salt, which is the key component of the e-liquid, is usually formed by the reaction of nicotine and organic acid. The solid atomizing cartridge also contains the nicotine salt. When the nicotine salt is atomized in the above two electronic atomizers, both the nicotine and organic acid will enter the human mouth cavity, nasal cavity, throat, intestinal tract, blood, lungs and brain with the atomized aerosol. Studies have proved that some organic acids are extremely harmful to the human body. For example, benzoic acid can irritate the skin and respiratory system, especially damage the mouth and cause severe coughing, and even damage the eyes; The minimum lethal dose of oxalic acid to humans is 71 mg/kg. The symptoms of poisoning after inhaling oxalic acid vapor in the human body are: extreme weakness, nasal mucosal ulcers, general pain, vomiting, etc. However, it is reported that benzoic acid and oxalic acid are the organic acids commonly used by nicotine salts in e-cigarettes. Furtherly, nicotine salts synthesized from other “safe” organic acids are not really safe. The metabolic organs, mechanisms and duration of metabolism of organic acids and nicotine in the human body are very different. Organic acids will destroy the acid-base balance of the human body and cause “acidosis” and other diseases; the human body is usually “acid excess”, and the organic acid in nicotine salt will undoubtedly aggravate the acid excess in the human body, and when inhaling the e-liquid containing nicotine salt by organic acid, the smoke will also be accompanied for a indescribable “sour smell”, which reduces the user's experience of e-cigarettes.

So far, there are only two existence modes of nicotine in all e-cigarette liquids and solid atomizing cartridges, namely free nicotine and organic acid nicotine salt. Free nicotine is volatile, unstable, irritating, and throat-choking, while the organic acid nicotine salt enters the human body during atomization, causing problems such as “acidosis”, “acid excess” and “sour smell”.

In addition, in order to decrease the addiction of nicotine, decreasing the concentration of nicotine in e-cigarettes has become a trend of global policy and regulation; However, users complain that e-cigarettes with low nicotine concentration have slow satisfaction of addiction, weak throat sensation, and poor suction experience, which reduces the competitiveness of e-cigarettes comparing to traditional tobacco and will cause users to choose the traditional tobacco again.

### Contents of the Invention

The main technical problem to be solved by the present invention is to provide a reaction compound of alkaloid and phenol, an e-liquid, an atomizing cartridge, and an electronic atomizer, which can eliminate the original irritating smell of alkaloids, and improve throat comfortable and suction experience.

In order to solve the above-mentioned technical problem, a technical scheme adopted by the present invention is to provide a reaction compound of alkaloid and phenol, including: a combined state R—PH—O—N—I obtained by chemical reaction of alkaloid and phenol, and the general formula of the chemical reaction is R—PH—OH+N—I→R—PH—O—N—I, wherein R—PH—OH is a reactant phenol, and N—I is a reactant alkaloid, and R— represents at least one of a hydrocarbon group, an aromatic group and a hydrogen atom, and PH represents at least one of phenyl ring aromatic hydrocarbon nucleus and fused benzene ring aromatic hydrocarbon nucleus, and —OH represents at least one hydroxyl group directly connected to the aromatic hydrocarbon nucleus, and N represents at least one nitrogen atom, and —I represents at least one of hydrocarbon group and aromatic group, and O represents oxygen atom. Unexpectedly: the phenolic hydroxyl can eliminate the original irritating smell of the alkaloid through the chemical reaction.

In a preferred embodiment of the present invention, the alkaloid is a combination of at least one of betaine, theophylline, capsaicin, arecoline, caffeine, piperine, pepperine, nicotine, theobromine, leonurine, hygrine, red cousidine, sophora alkaloid, integerrimine, monocrotaline, nicotine, lobeline, lobelialine, expectorantine, sanguinarine, maytansine, small berberine, berberine, tetrandrine, African tetrandrine, sanguinarine citrate, reserpine, pilocarpine, atropine, hyoscyamine, scopolamine, halosine, solanine, belladonna alkaline, solanine, fritillary base, fritillary base, aconitine, delphinium, galanthamine, tripteryline, tripteryline, venomine, anabasine, coptisine, North American berberine, sinomenine, magnolialine, erpentine, n-methylcytisine, anagerline, bapufoline, matrine, oxymatrine, hydroxymatrine, dehydrogenation matrine, matrine, ginseng alkaloid, evodia alkaloid, schiff base, vinblastine, vincristine, cinchonaline, strychnine, rhynchophylline, isatidine, macaine, moringa alkaloid, kudzu root carnitine, guarana alkaloid, ganoderma alkaloid, L-carnitine, lotus leaf alkaloid, cactus alkaloid, seabuckthorn alkaloid, plum essence, papaya alkaloid, chive alkaloid, epimedium alkaloid, cynomex alkaloid, cistanche alkaloid, actinidinine, lycorine, aloe alkaloid, mulberry leaf carnitine, mulberry leaf alkaloid, ginger alkaloid, golden clover alkaloid, hippocampus choline, angelica, chuanbei alkaloid, ergot alkaloid, ergometrine, pilocarpine, sophocarpine, bapufoline, hacmatine, dauricine, corydine aureus, genus alkaloid, silkworm excrement alkaloid, diconazole alkaloid, emetine, qingfengteng alkaloid, chuanxiong alkaloid, oat alkaloid, ryegrass alkaloid, celandine total alkaloids, dandelion alkaloid, corn silk alkaloid, seabuckthorn alkaloid, cocklebur alkaloid, jatrorrhizine, hibiscus alkaloid, camptothecin, phellodendron, quinoa alkaloid, and the phenol is a combination of at least one of catechol, tea polyphenol, maltol, ethyl maltol, paconol, phenol, cannabinol, cannabidiol, cannabigerol, cannabichromene, cannabidiol, alkylphenol, acetaminophen, nitrophenol, naphthol, chlorophenol, biquinone, resorcinol, hydroquinone, pyrogallol, phloroglucinol, pyroglucinol, cresol, tocopherol, tocotrienol, proanthocyanidin polyphenol, honey polyphenol, olive polyphenol, chocolate polyphenol, pine polyphenol, eugenol, isocugenol, rhubarb phenol, Ginkgo biloba polyphenol, algae polyphenol, seaweed polyphenol, brown algae polyphenol, raspberry polyphenol, santalol, muskol, thymol, agarol, star anise polyphenol, star anise phenol, thymol, eucalyptus phenol, oregano phenol, carvacrol, carvacrol, zingerol, gossypol, agrimonol, bakuchiol, ligninol, cardanol, geranol, schisandrol, cholesterol, chlorine myol, 4-aminophenol, orcinol, clomiphene, folinol, phenylselenol, dendrobium phenol, kaempferol, taxol, honokiol, magnolol, total magnolol, sesamol, apple polyphenol, peach polyphenol, pear polyphenol, grapefruit polyphenol, bayberry polyphenol, citrus polyphenol, macaque peach polyphenol, banana polyphenol, orange polyphenol, papaya polyphenol, cantaloupe polyphenol, chestnut shell polyphenol, chestnut bract polyphenol, loquat polyphenol, cocoa polyphenol, cocoa bean polyphenol, monk fruit polyphenol, lotus seed polyphenol, potato polyphenol, jujube polyphenol, winter jujube polyphenol, sandy date polyphenol, jujube polyphenol, garlic polyphenol, onion polyphenol, corn polyphenol, yam polyphenol, sorghum polyphenol, peanut kernel polyphenol, peanut red coat polyphenol, grape polyphenol, grapefruit polyphenol, red wine polyphenol, mung bean polyphenol, cowpea polyphenol, wolfberry polyphenol, sunflower seed polyphenol, lily polyphenol, sealwort polyphenol, polygonatum polyphenol, camellia oleifera polyphenol, white fungus polyphenol, fungus polyphenol, lotus root polyphenol, pueraria polyphenol, fennel polyphenol, thistle polyphenol, evening primrose polyphenol, coriander polyphenol, cumin polyphenol, lavender polyphenol, rose polyphenol, chrysanthemum polyphenol, jasmine polyphenol, sophora polyphenol, osmanthus polyphenol, gardenia polyphenol, olive polyphenol, plum polyphenol, guaiacol, 4-methylguaiacol, 2-isopropylphenol, 2-methoxy-4-vinylphenol, dimethoxyphenol, and 3,4-dimethylphenol.

Unexpectedly, the reaction compound of “alkaloid-phenol” prepared by the joint chemical reaction of several alkaloids such as nicotine, piperine, ginger alkaloids and several phenols, comparing to the reaction compound prepared by a single nicotine and a single phenol under the same conditions, can enable users to obtain a faster, more enjoyable, and satisfying suction experience. This phenomenon is presumed to be that alkaloids such as piperine and ginger alkaloids can also promote the secretion of dopamine in the human brain, and also have physiological effects such as hotting, accelerating heartbeat, and accelerating blood circulation, which is conducive to improving the transmission efficiency of nicotine. Alkaloids such as piperine and ginger alkaloids may have a synergistic enhancement effect on the physiological reward mechanism of human blood brain barrier-nicotine-acetylcholine-dopamine.

When preparing the reaction compound of “alkaloid-phenol”, the alkaloid added to the reaction vessel is at least one of the extracts from animal or plant, purified products and synthetic products containing the alkaloid. These three types of raw materials of alkaloids can be purchased on the market, for example, tobacco extract of nicotine, plant purified nicotine, artificially synthesized nicotine, etc. The inventors have verified through a large number of experiments that the above-mentioned several alkaloids can indeed together chemically react with the several phenols to form an reaction compound of “alkaloid-phenol”, which can improve the inhalation experience of the e-liquid.

Unexpectedly, the reaction compound of “alkaloid-phenol” prepared by the joint chemical reaction of several phenols such as tea polyphenol, cannabidiol, eugenol and several alkaloids, comparing to the reaction compound prepared by a single nicotine and a single phenol under the same conditions, can enable users to obtain a fragrant, pleasant and soothing suction experience. This phenomenon is presumed to be that phenols such as tea polyphenol, cannabidiol, and eugenol can also promote the secretion of dopamine in the human brain, and also have physiological effects such as relieving anxiety, calming the nerves and releasing stress, promoting body fluids and quenching thirst, etc, which are beneficial for improving the user's suction experience. When preparing the reaction compound of “alkaloid-phenol”, the phenol added to the reaction vessel is at least one of the extracts from animal or plant, purified products and artificial products containing the phenol. These three types of raw materials of phenol can be purchased in the market, for example, hemp extract of cannabidiol, plant purified cannabidiol, artificially synthesized cannabidiol, etc. The inventors have verified through a large number of experiments that the above-mentioned several phenols can indeed together chemically react with the several alkaloids to form an reaction compound of “alkaloid-phenol”, which can improve the inhalation experience of the e-liquid.

In order to solve the above-mentioned problem, another technical solution adopted in the present invention is: a kind of production method of the reaction compound of alkaloid and phenol is provided, comprising the following steps:
add reactant alkaloid and reactant phenol in reaction vessel, carry out heating;
heat and stir continuously for 0.1-6000 minutes at a temperature of 40° C. to 300° C., and then the reaction compound of alkaloid and phenol is prepared.

Furtherly, when heating and stirring alkaloid and phenolic raw materials to prepare reaction compound of alkaloid and phenol, an appropriate amount of propylene glycol, water, butanediol, ethanol, medium-chain triglycerides and other non-reactive substances can be added in the reaction vessel as a liquid medium to quickly dissolve the raw materials of alkaloids and phenols, which will also enter the e-liquid with the reaction compound of alkaloid and phenol to be an e-liquid solvent or be mixed into the solid atomizing cartridge. Only liquid alkaloid reactants in such as nicotine can also directly dissolve the phenolic raw materials without adding these liquid medium in the reaction vessel.

Although alkaloid and phenol can also undergo a chemical reaction under continuous stirring to produce a combined “alkaloid-phenol” at a temperature lower than 40° C., the reaction takes a long time and the production efficiency is low. The chemical reaction between alkaloid and phenol is fast at 40° C. to 300° C., and a chemical reaction compound of combined state “alkaloid-phenol” can be obtained after continuous stirring for 0.1-6000 minutes. When the temperature is higher than 300° C., under continuous stirring, although the reaction compound of combined state “alkaloid-phenol” can also be fast prepared by the chemical reactions of alkaloid and phenol, the physiological activity of the prepared “alkaloid-phenol” in the e-liquid tends to decline and deteriorate.

Unexpectedly, if the alkaloid and phenol are not mixed, stirred and heated to promote a chemical reaction to prepare the reaction compound of “alkaloid-phenol”, and the non-reactive liquid medium such as propylene glycol can also be added to prepare an e-liquid, and the phenolic raw material is added to the mixture of main solvent, essence, nicotine and organic acid nicotine salt till the e-liquid is prepared at room temperature, although there are also two components of alkaloid and phenol in the obtained e-liquid, the alkaloid and phenol are independent of each other to be a free state but not the combined state, for example, the alkaloid in the nicotine salt has been combined with the organic acid to form a salt, and it is difficult to combine with the phenol again, and the difference in boiling points between the alkaloid and the phenol causes the atomized aerosol to be rough and uneven, so the user's suction experience is not good. This phenomenon can also prove that there is indeed a combined state “alkaloid-phenol” in the chemical reaction compound of alkaloid and phenol of the present invention. In the combined state “alkaloid-phenol”, phenol can not only eliminate the original irritation of the alkaloid, but also can balance atomized aerosol by combined with alkaloids to better the suction experience.

Furtherly, the combined state “alkaloid-phenol” in the reaction compound of alkaloid and phenol of the present invention also can be proved by two phenomena: when the phenol and alkaloid liquid are directly mixed and stirred in a round bottom flask at room temperature, the bottom flask will generate heat, indicating that the two compounds are undergoing a combination reaction to generate a new substance, usually the combination reaction will be exothermic. Dropping 0.1 ml of alkaloid liquid such as nicotine on the table, we can smell the strong nicotine pungent smell immediately, and the nicotine will evaporate in about 10 minutes; but dropping 0.1 ml of the reaction compound of nicotine and tea polyphenol on the table, almost no pungent smell, and the “nicotine-tea polyphenol” droplets still stay on the table after 3 hours. These phenomena indicate that there is indeed a combined state “nicotine-tea polyphenol” in the reaction compound of nicotine and tea polyphenol. Tea polyphenol not only eliminate the original irritation of nicotine, but also combines with nicotine to change the unstable and volatile nature of nicotine.

The reaction vessel is a conventional chemical reaction synthesis container, including but not limited to glass reactors, enamel-lined reactors, plastic-lined reactors and metal reactors, etc. The heating methods include but not limited to resistance heating, microwave heating and ultrasonic heating, etc.

Unexpectedly, alkaloid nitrogen atom and phenolic hydroxyl do not have to be chemically covalently bonded in a molar ratio of 1:1 to form an reaction compound of “alkaloid-phenol”. Alkaloids and phenols can be one-to-many or many-to-many in the chemical reaction to produce the combined state “alkaloid-phenol”, and even within the ratio range of moles of nitrogen atoms of the raw material alkaloids to moles of phenolic hydroxyl is (0.0001-10000)/1, the combined state “alkaloid-phenol” can be produced in the reaction compound. The theoretical explanation of this phenomenon is: during the stereochemical reaction process, affected by the microcosmic effects of electron jumping in a huge space and electric field radiation, the lone electron pair of the alkaloid nitrogen atom will attract multiple phenolic hydroxyl around it to form a combined state “alkaloid-phenol”, and the phenolic hydroxyl can also attract multiple alkaloid nitrogen atoms to form a combined state “alkaloid-phenol”. The chemical bond energy between the alkaloid nitrogen atom and the phenolic hydroxyl in the combined state “alkaloid-phenol” is slightly weaker than the covalent chemical bond but far greater than the intermolecular force (van der Waals attractiveness). This weak bond is conducive to the one-to-many or many-to-many chemical reaction between alkaloids and phenol molecules in three-dimensional space to form combined state “alkaloid-phenol” clusters, wherein the spatial structure and chemical combination state are very conducive to the “alkaloid-phenol” in the e-liquid, in the atomization core, in the aerosol channel, and in the mouth cavity of the human body, while the combined state “alkaloid-phenol” can be separated into “alkaloid” and “phenol” in time under the influence of saliva, mucous membranes, blood, etc., after entering the human body, and absorbed and metabolized by different organs; the reaction compound of “alkaloid-phenol” can eliminate the original irritating smell of alkaloids, and improve the transmission efficiency and suction experience of alkaloids, which is in line with the e-cigarettes using condition.

In order to solve the above-mentioned problem, another technical solution adopted by the present invention is to provide an e-liquid, which contains the above-mentioned reaction compound of alkaloid and phenol.

In a preferred embodiment of the present invention, solvent and essence are also included. After uniformly mixing the reaction compound of alkaloid and phenol with solvent and essence to form an e-liquid, it can significantly weaken the alkaloid smell, balance the aerosol, and improve the softness of the throat. So that, the users can quickly get satisfaction of addiction, can solve the problems of nicotine salt of “acidosis”, “acid excess” and “sour smell”, as well as increase the special aroma introduced by non-essences, which significantly improves the user's suction experience of e-cigarettes.

In a preferred embodiment of the present invention, the weight ratio of the reaction compound of alkaloid and phenol in the e-liquid is 0.001% to 96%, and the weight ratio of the solvent in the e-liquid is 3% to 98.999%, and the weight ratio of the essence in the e-liquid is 1% to 30%.

In a preferred embodiment of the present invention, the solvent is a combination of at least one of propylene glycol, glycerin, water, butylene glycol, ethanol, and medium-chain triglyceride, and the essence is a combination of at least one of sweetener, cooling agent, fruit essence, tobacco essence, meat essence, mint essence, food essence, sweeteners such as sucralose, neotame, aspartame, alitame, etc., cooling agents such as menthol, etc. The solvents and essences are commodities that can be directly purchased on the market and are preferably food grade. Furtherly, in order to meet the needs of some users, some functional health-care substances can also be added to the e-liquid as essence components.

In order to solve the above-mentioned problem, another technical solution adopted by the present invention is to provide an atomizing cartridge, which contains the above-mentioned reaction compound of alkaloid and phenol.

In a preferred embodiment of the present invention, the atomizing cartridge adopts at least one of a liquid atomizing cartridge and a solid atomizing cartridge. The liquid atomizing cartridge using the e-liquid containing the reaction compound of “alkaloid-phenol”, and the solid atomizing cartridge is prepared by dispersing the reaction compound of “alkaloid-phenol” on a carrier, and the carrier is in the form of granule, flake, colloid, etc., and the carrier material is dried plant granules or artificial fibers.

The liquid atomizing cartridge of the present invention can be produced by conventional processes and equipment:
Use a mold and an injection molding machine to thermally mold PCTG plastic into the shell of the atomizing cartridge, inject an appropriate amount of the above-mentioned e-liquid containing “alkaloid-phenol” into the atomizing cartridge, and plug the silicone sealing ring, atomizing core, base and other components into the atomizing cartridge to seal the e-liquid, so that the close-type liquid atomizing cartridge is obtained;
The open-type liquid atomizing cartridge is prepared by injecting an appropriate amount of the above-mentioned e-liquid containing “alkaloid-phenol” into an open atomizing cartridge and sealing the injection hole.

The solid atomizing cartridge of the present invention can also be produced by conventional processes and equipment:
Mix an appropriate amount of reaction compound of “alkaloid-phenol” with dried plant granules or artificial fibers, and extrude with a screw, spheroidize, shot blast, dry, and sieve to obtain fuming particles;
Mix an appropriate amount of reaction compound of “alkaloid-phenol” with dried plant granules or artificial fibers evenly, roll, dry, and demould to prepare fuming flakes;
Dried plant granules or artificial fibers are soaked in water for pulping and made into a fiber sheet by a paper machine, and the liquid reaction compound of “alkaloid-phenol” is evenly coated on the surface of the fiber sheet, dried and cut to obtain fume flakes;
Uniformly doping the liquid reaction compound of “alkaloid-phenol” into silica gel to obtain fuming colloid;
A fuming section filled with the above-mentioned fuming particles, fuming flakes or fuming colloid, a cooling section of food-grade silica gel, and a filter section are packaged together into one body by using packaging materials such as tipping paper to obtain a solid atomizing cartridge.

In order to solve the above problem, another technical solution adopted by the present invention is to provide an electronic atomizer, the electronic atomizer contains the reaction compound of alkaloid and phenol, and the electronic atomizer is at least one of a vapor electronic atomizer that consumes liquid atomizing cartridges and a HNB electronic atomizer that consumes solid atomizing cartridges.

The beneficial effect of the present invention is: a kind of reaction compound of alkaloid and phenol, an e-liquid, an atomizing cartridge and an electronic atomizer pointed out by the present invention have the following effects:

The synthesis reaction of alkaloid and phenol is different from the nicotine salt technology. The nicotine salt technology relies on the reaction of carboxylic acid group —COOH and nicotine to obtain a combined state “nicotine-acid”, while the present invention relies on the chemical reaction of phenolic hydroxyl —OH and alkaloid to obtain a combined state “alkaloid-phenol”; when chemically reacting with alkaloid, the functional groups of acid and phenol are different, as well as the structure of products are also different.

The reaction compound of alkaloid and phenol in the present invention obtains a combined state “alkaloid-phenol”, which betters the original throat-stimulating taste of alkaloids, is beneficial to the mouth inhalation, and produces a special fragrant aroma in the e-liquid aerosol which is different with the fragrances of essences;

The combined state “alkaloid-phenol” with a synergistic enhancement effect of several alkaloids and several phenols enables users to obtain a faster, more pleasant and satisfying suction experience under the same conditions;

The present invention utilizes alkaloid and phenol to prepare an e-liquid containing reaction compound of “alkaloid-phenol”, does not contain organic acids and nicotine salts, and without “acidosis”, “acid excess”, “sour odor” and other issues;

Both the atomizing cartridge and the electronic atomizer of the present invention contain the reaction compound of alkaloid and phenol, that is the combined state “alkaloid-phenol”, which improves the user's suction experience.

### BRIEF DESCRIPTION OF THE DRAWINGS

In order to more clearly illustrate the technical solutions in the embodiments of the present invention, the accompanying drawings that need to be used in the description of the embodiments will be briefly described below, and it is obvious that the accompanying drawings in the following description are only some of the embodiments of the present invention, and other drawings can be obtained by those skilled in the art based on these accompanying drawings without creative work, wherein:

FIG. 1 is the chemical reaction formula of the combined state “nicotine-cannabidiol” obtained from a reaction of nicotine and cannabidiol in the reaction compound of alkaloid and phenol of the present invention.

FIG. 2 is the chemical reaction formula of the combined state “arecoline-cannabigerol” obtained from a reaction of arecoline and cannabigerol in the reaction compound of alkaloid and phenol of the present invention.

### MODE OF CARRYING OUT THE INVENTION

The following will clearly and completely describe the technical solutions in the embodiments of the present invention. Obviously, the described embodiments are only some of the embodiments of the present invention, rather than all the embodiments. Based on the embodiments of the present invention, all other embodiments obtained by person of ordinary skill in the art without making creative effort belong to the protection scope of the present invention.

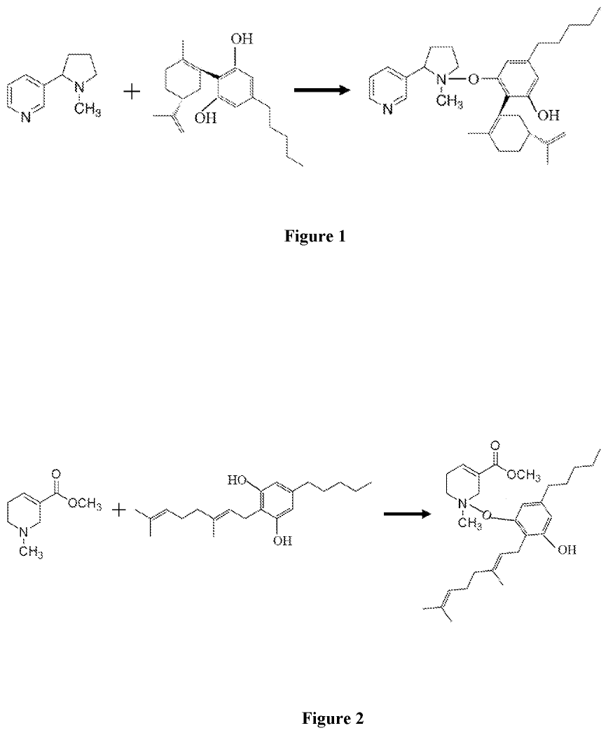

Referring to FIG. 1 and FIG. 2, the functional group characteristics and chemical properties of phenols are significantly different from those of organic acids, alcohols, aldehydes, ketones, ethers, esters, hydrocarbons, etc. Therefore, there is a fundamental difference between the “alkaloid-phenol”, synthesized by the reaction of alkaloid and phenol in the present invention, and the nicotine salt formed by nicotine and organic acid.

Embodiments of the invention include:

### Comparative Example 1

In a glass beaker, add 29.57 g of propylene glycol, 0.50 g of plant nicotine liquid with a purity of 99.5%, 1.54 g of mung bean essence, 0.14 g of sucralose, 1 g of menthol cooling agent, 16 g of plant glycerin and 1 g of cannabidiol powder with a purity of 98%, and mix evenly to obtain an e-liquid containing nicotine, cannabidiol, essence and solvent, and the mass concentration of nicotine is 1%, labeled as “A1 e-liquid”, wherein the total mass ratio of alkaloid and phenol is 2.97% in the e-liquid.

Take 2 ml of the above-mentioned “A1 e-liquid”, inject it into an e-cigarette liquid cartridge by using conventional processes and equipment, then obtain a vapor electronic atomizer comparison sample, which is marked as “A1 atomizer”.

### Comparative Example 2

In a glass beaker, sequentially add 30.506 g of propylene glycol, 0.974 g of synthetic nicotine liquid with a purity of 99.9%, 0.316 g of arecoline with a purity of 98% and 0.638 g of cannabigerol powder with a purity of 99%, and mix evenly to obtain an e-liquid containing nicotine, arecoline, cannabigerol and propylene glycol, labeled as “B1 e-liquid”, in which the mass concentration of nicotine is about 3%, and the total mass ratio of alkaloid and phenol in the e-liquid is 5.9%.

According to the conventional preparation processes and equipment of HNB fuming particles, dope a certain amount of the above-mentioned “B1 e-liquid” into the fuming sheet, and use the matching rod to obtain a HNB electronic atomizer comparison sample, which is marked as “B1 atomizer”.

### Comparative Example 3

In a glass beaker, add 2.4187 g of propylene glycol, 0.0813 g of water, 0.00147 g of theophylline powder with a purity of 99.5%, 25.727 g of carvacrol liquid with a purity of 95%, 21.771 g of chrysophanol powder with a purity of 95%, and 0.85 g of tobacco essence, 0.05 g of sucralose, 0.1 g of menthol cooling agent, and 1.77 g of vegetable glycerin, and mix evenly to obtain an e-liquid, which is marked as “C1 e-liquid”, wherein the total mass ratio of alkaloid and phenols in the e-liquid is 90%.

According to the conventional preparation processes and equipment of HNB fuming particles, dope a certain amount of the above-mentioned “C1 e-liquid” into the fuming particles, and use the matching rod to obtain a HNB electronic atomizer comparison sample, which is marked as “C1 atomizer”.

### Comparative Example 4

In a glass beaker, add 265.773 g of plant-extracted nicotine liquid with a purity of 99.7%, 9.461 g of hygrine liquid with a purity of 99.5%, 0.016 g of phenol with a purity of 98%, and 0.0242 g of sesamol powder with a purity of 95%, and mix evenly by stirring to obtain a mixed solution; add 0.001 g of the above mixed solution, 2 g of sweet orange essence, 2 g of blood orange essence, 0.99 g of aspartame, 0.009 g of neotame, 5 g of menthol cooling agent, 40 g of propylene glycol and 50 g of glycerol in a conical flask, and mix uniformly to obtain an e-liquid, which is labeled as “D1 e-liquid”, wherein the total mass ratio of alkaloids and phenols in the e-liquid is 0.001%.

Take 2 ml of the above-mentioned “D1 atomizing liquid”, inject it into a disposable e-cigarette by using conventional processes and equipment, then obtain a vapor electronic atomizer comparison sample, which is marked as “D1 atomizer”.

### Comparative Example 5

In a glass beaker, add 779.849 g of arecoline with a purity of 99.5%, 30.495 g of caffeine powder with a purity of 99.5%, 28.435 g of theobromine powder with a purity of 99%, 0.05 g of cannabidiol powder with a purity of 98%, and 0.054 g of eugenol powder with a purity of 95%, 0.047 g of carvacrol liquid with a purity of 99.5%, and 0.018 g of catechol powder with a purity of 95%, and mix evenly by stirring to obtain a mixture solution. In an erlenmeyer flask, add 45 g of the above mixed solution, and then add 3 g of watermelon essence, 1 g of passion fruit essence, 0.5 g of alitame, 0.5 g of neotame, 5 g of menthol cooling agent, 20 g of propylene glycol and 25 g of glycerol, and mix evenly to obtain an e-liquid, labeled as “E1 e-liquid”, wherein the total mass ratio of alkaloids and phenols in the e-liquid is 45%.

Take 2 ml of the above-mentioned “E1 e-liquid”, and inject it into an e-cigarette using conventional processes and equipment, then obtain a vapor electronic atomizer comparison sample, which is marked as “E1 atomizer”.

### Comparative Example 6

In a three-necked round-bottomed flask, add 2 g of tobacco-extracted liquid containing nicotine with the total number of detected alkaloid nitrogen atoms about 0.01 moles, 0.62 g of benzoic acid powder with a purity of 98%, and 100 g of medium-chain triglycerides, stir and react for 60 minutes at 100° C. to prepare nicotine benzoate. After cooling to room temperature, add 50 g of cannabis polyphenol and mix evenly to obtain a mixed solution. Get 5 parts by weight of the mixed solution, add 45 parts by weight of propylene glycol, 50 parts by weight of propylene glycol to obtain an e-liquid of 100 parts by weight, labeled as “F1 e-liquid”, wherein the total mass ratio of alkaloids and phenols in the e-liquid is 1.7%.

Take 10 ml of the above-mentioned “F1 e-liquid” containing nicotine benzoate and cannabis polyphenol, and inject it into the cartridge of a large e-cigarette with a power of 70 watts using conventional processes and equipment, then get a vapor electronic atomizer containing alkaloids and phenols, which is marked as “F1 atomizer”.

### Comparative Example 7

Take a nicotine-oxalate salt which is prepared by 81.196 g of synthetic nicotine liquid with a purity of 99.9% and 23 g of oxalic acid, 16.55 g of arecoline extract, 30 g of tea polyphenol, and 21.08 g of lily polyphenol extract, and mix evenly to obtain a mixture of nicotine-oxalate salt, arecoline extract, tea polyphenol, and lily polyphenol.

Dope a certain amount of the above mixture into the fuming colloid to make a solid atomizing cartridge according to the conventional preparation processes and equipment of HNB fuming particles, use the matching rod to obtain a HNB electronic atomizer comparison sample, which is marked as “G1 atomizer”.

### Embodiment 1

In a polytetrafluoroethylene plastic container, add 95.16 g of propylene glycol as the reaction medium, under magnetic stirring, and add reactants of 1.63 g of plant-extracted nicotine liquid with a purity of 99.5% (C₁₀H₁₄N₂, about 0.02 moles of nitrogen atoms) and 3.21 g of cannabidiol powder with a purity of 98% (C₂₁H₃₀O₂, about 0.02 moles of phenolic hydroxyl), the moles number of alkaloid nitrogen atoms to the moles number of phenolic hydroxyl is 1/1. Seal the polytetrafluoroethylene container into a hydrothermal reaction kettle and put it into a magnetic stirrer with a round sleeve temperature control, turn on the heating, set a continuous stirring speed of 300 rps and heat at a constant temperature of 150° ° C. for 120 minutes, and then turn off the power and let it cool naturally. Take out the PTFE container. A mixture of reaction compound of nicotine-cannabidiol as combined state “alkaloid-phenol” and propylene glycol is obtained, in which the mass ratio of nicotine is about 1.62%.

In a glass beaker, add 30.86 g of the above-mentioned mixture containing reaction compound of nicotine-cannabidiol and propylene glycol, and then add 1.54 g of mung bean essence, 0.14 g of sucralose, 1 g of menthol cooling agent, 16 g of plant glycerol, stir evenly to get an e-liquid containing the reaction compound of “alkaloid-phenol”, labeled as “A2 e-liquid”, in which the mass ratio of nicotine in the e-liquid is 1%, and the mass ratio of the reaction compound of “alkaloid-phenol” in the e-liquid is 2.97%.

Take 2 ml of the above-mentioned “A2 e-liquid” containing the reaction compound of nicotine-cannabidiol, inject it into an liquid atomizing cartridge using conventional processes and equipment, and obtain a vapor electronic atomizer containing the reaction compound of alkaloid and phenol, which is marked as “A2 atomizer”.

### Embodiment 2

In a three-necked round-bottomed flask, add 150.53 g of propylene glycol as the reaction medium, and 4.87 g of synthetic nicotine liquid with a purity of 99.9% (C₁₀H₁₄N₂, about 0.06 moles of nitrogen atoms), 1.58 arecoline with a purity of 98% (C₈H₁₃NO₂, about 0.01 moles of nitrogen atoms), 3.19 g of cannabigerol powder with a purity of 99% (C₂₁H₃₂O₂, about 0.02 moles of phenolic hydroxyl), the ratio of mole number of nitrogen atoms of alkaloids to mole number of phenolic hydroxyl is 3.5/1, use a circular sleeve heater to control the reaction temperature in the round-bottomed flask to 40° C., continue heating and stirring for 6000 minutes, turn off the power and cool naturally, and then obtain a mixture solution of the reaction compound of “alkaloid-phenol” containing combined state (nicotine+arecoline)-cannabigerol and propylene glycol, add 1 g of sucralose sweetener and 1 g of menthol cooling agent essence, labeled as “B2 e-liquid”, wherein the mass ratio of nicotine in the e-liquid is 3%, and the mass ratio of the reaction compound of “alkaloid-phenol” in the e-liquid is 5.9%.

According to the conventional preparation processes and equipment of HNB fuming flakes, dope an appropriate amount of “B2 e-liquid” containing the above-mentioned reaction compound of (nicotine+arecoline)-cannabigerol into the fuming flakes to obtain a solid atomizing cartridge. Using the matching rod, a HNB electronic atomizer containing the reaction compound of alkaloid and phenol is obtained, which is marked as “B2 atomizer”.

### Embodiment 3

In a polytetrafluoroethylene container, add 29.74 g of propylene glycol and 1 g of water as the mixed reaction medium, under magnetic stirring, sequentially add the reactants of 0.0181 g of theophylline powder with a purity of 99.5% (C₇H₈N₄O₂, about 0.0004 moles of nitrogen atom), 316.24 g of carvacrol liquid with a purity of 95% (C₁₀H₁₄O, about 2 moles of phenolic hydroxyl) and 267.62 g of chrysophanol powder with a purity of 95% (C₁₅H₁₀O₄, about 2 moles of phenolic hydroxyl), the ratio of moles number of alkaloid nitrogen atoms to moles number of phenolic hydroxyl is 0.0001/1, seal the polytetrafluoroethylene container in a hydrothermal reaction kettle, and seal the reaction kettle and then put it into a temperature-controlled magnetic stirrer, turn on the heating, set a continuous stirring at speed of 300 rps and heat at a constant temperature of 130° C. for 20 minutes, turn off the power and cool naturally, then take out the polytetrafluoroethylene container, and obtain a mixture containing the reaction compound of “alkaloid-phenol” of theophylline-(carvacrol+chrysophanol), propylene glycol and water, and the mass ratio of “alkaloid-phenol” is about 95%.

In a glass beaker, add 50 g of the above-mentioned mixture containing theophylline-(carvacrol+chrysophanol), propylene glycol and water, and then add 0.85 g of tobacco essence, 0.05 g of sucralose, 0.1 g of menthol cooling agent, 1.77 g of plant glycerin, and stir evenly to obtain an e-liquid containing the reaction compound of “alkaloid-phenol”, wherein the mass ratio of the reaction compound of “alkaloid-phenol” in the e-liquid is 90%, which is marked as “C2 e-liquid”.

According to the conventional preparation processes and equipment of HNB fuming particles, dope an appropriate amount of the above-mentioned “C2 e-liquid” containing the reaction compound of theophylline-(carvacrol+chrysophanol) into the fuming particles to obtain a solid atomizing cartridge. Using the matching rod, and then obtain a HNB electronic atomizer containing the reaction compound of alkaloid and phenol, which is marked as “C2 atomizer”.

### Embodiment 4

In a polytetrafluoroethylene container, under a magnetic stirring, sequentially add the reactants of 7973.189 g of plant-extracted nicotine liquid with a purity of 99.7% (C₁₀H₁₄N₂, about 98 moles of nitrogen atoms), 283.839 g of hygrine liquid with a purity of 99.5% (C₈H₁₅NO, about 2 moles of nitrogen atoms), 0.4802 g of phenol with a purity of 98% (C₆H₅OH, about 0.005 moles of phenolic hydroxyl) and 0.7269 g of sesamol powder with a purity of 95% (C₇H₆O₃, about 0.005 moles of phenolic hydroxyl), the ratio of moles number of alkaloid nitrogen atoms to the moles number of phenolic hydroxyl is 10000/1, and then seal the polytetrafluoroethylene container into a hydrothermal reaction kettle; seal the reaction kettle and put it into a temperature-controlled magnetic stirrer, turn on the heating, set a continuous stirring at speed of 500 rps and heat at a constant temperature of 120° C. for 90 minutes, turn off the power and naturally cool down, then take out the polytetrafluoroethylene container till the pressure is reduced, and obtain a liquid reaction compound of combined state “alkaloid-phenol’ of (nicotine+hygrine)-(phenol+sesamol).

In a glass beaker, add 0.01 g of the above-mentioned liquid reaction compound of (nicotine+hygrine)-(phenol+sesamol), and then add 20 g of sweet orange essence, 20 g of blood orange essence, and 9.9 g of aspartame, 0.09 g of neotame, 50 g of menthol cooling agent, 400 g of propylene glycol and 500 g of glycerol, stir evenly to obtain an e-liquid containing the reaction compound of “alkaloid-phenol” of (nicotine+coustraline)-(phenol+sesamcol), wherein the mass ratio of the reaction compound of “alkaloid-phenol” in the e-liquid is 0.001%, which is marked as “D2 e-liquid”.

Take 2 ml of the above-mentioned “D2 e-liquid” containing the reaction compound of (nicotine+coustraline)-(phenol+sesamcol), inject it into a disposable e-cigarette by using conventional processes and equipment, and obtain a vapor electronic atomizer containing the reactive composition of alkaloid and phenol, which is marked as “D2 Atomizer”.

### Embodiment 5

In a microwave reactor, add reactants of 6238.794 g of arecoline with a purity of 99.5% (C₈H₁₃NO₂, about 40 moles of nitrogen atoms), 243.957 g of caffeine powder with a purity of 99.5% (C₈H₁₀N₄O₂, about 5 moles of nitrogen atoms), 227.479 g of theobromine powder with a purity of 99% (C₇H₈N₄O₂, about 5 moles of nitrogen atoms), 0.401 g of cannabidiol powder with a purity of 98% (C₂₁H₃₀O₂, about 0.0025 moles of phenolic hydroxyl), 0.432 g of eugenol powder with a purity of 95% (C₁₀H₁₂O₂, about 0.0025 moles of phenolic hydroxyl), 0.377 g of carvacrol liquid with a purity of 99.5% (C₁₀H₁₄O, about 0.0025 moles of phenolic hydroxyl) and 0.1447 g of catechol powder with a purity of 95% (C₆H₆O₂, about 0.0025 moles of phenolic hydroxyl), and the ratio of moles number of nitrogen atoms in alkaloids to moles number of phenolic hydroxyl is 5000/1, then seal the microwave reactor and turn on the microwave heater, the microwave frequency is 2×10⁹ hertz, the microwave power is 5 kilowatts and the stirring speed is 200 rps, and the microwave heating and stirring is continued for 0.1 minute, and the detected highest heating temperature is 260° C. Turn off the microwave power supply, open the microwave reactor after natural cooling and depressurization, and take out the liquid to obtain a reaction compound of “alkaloid-phenol” of combined state (arecoline+caffeine+theobromine)-(cannabidiol+eugenol+carvacrol+catechol).

In a glass beaker, add 45 g of the above-mentioned liquid reaction compound of (arecoline+caffeine+theobromine)-(cannabidiol+eugenol+carvacrol+catechol), then add 3 g of watermelon essence, 1 g passion fruit essence, 0.5 g alitame, 0.5 g neotame sweetener, 5 g menthol cooling agent, 20 g propylene glycol and 25 g glycerol, stir evenly to obtain an e-liquid containing the reaction compound of combined state “alkaloid-phenol” of (arecoline+caffeine+theobromine)-(cannabidiol+eugenol+carvacrol+catechol), wherein the mass ratio of reaction compound of “alkaloid-phenol” in the e-liquid is 45%, which is marked as “E2 e-liquid”.

Take 2 ml of “E2 e-liquid” containing the above mentioned reaction compound of (arecoline+caffeine+theobromine)-(cannabidiol+eugenol+carvacrol+catechol), and inject it into the an open type atomizing cartridge of e-cigarette by using conventional processes and equipment, a vapor electronic atomizer containing the reaction compound of alkaloid and phenol is obtained, which is marked as “E2 atomizer”.

### Embodiment 6

In a three-necked round-bottomed flask, add 2 g of tobacco-extracted liquid containing nicotine with about 0.01 mole of alkaloid nitrogen atoms by detection, and 50 g of cannabis polyphenol containing about 0.2 mole of phenolic hydroxyl by detection, and add 100 g of medium-chain triglycerides ester MCT as the reaction liquid medium, the ratio of moles of nitrogen atoms in alkaloids to moles of phenolic hydroxyl is 1/20, turn on the heating, stir and react at 80° C. for 60 minutes, and then obtain a reaction compound of combined state “alkaloid-phenol” of nicotine-cannabis polyphenol.

Get 5 parts by weight of the reaction compound, add 15 parts by weight of menthol cooling agent essence, 15 parts by weight of sucralose sweetener essence, 30 parts by weight of propylene glycol, and 35 parts by weight of glycerol to obtain an e-liquid of 100 parts by weight, labeled as “F2 e-liquid”, wherein the mass ratio of reaction compound of “alkaloid-phenol” in the e-liquid is 1.7%.

Take 10 ml of the above-mentioned “F2 e-liquid” containing the reaction compound of combined state “alkaloid-phenol” of nicotine-cannabis polyphenol, and inject it into the cartridge of a large e-cigarette with a power of 70 watts using conventional processes and equipment, then get a vapor electronic atomizer containing the reaction compound of alkaloid and phenol, which is marked as “F2 atomizer”.

### Embodiment 7

In an enamel-lined reaction kettle, add 81.196 g of synthetic nicotine liquid with a purity of 99.9% (C₁₀H₁₄N₂, about 1 mole of nitrogen atoms), 16.55 g of arecoline extract containing about 0.1 mole of nitrogen atoms by detection, and 30 g of tea polyphenol containing about 0.2 mole of phenolic hydroxyl by detection, 21.08 g of lily polyphenol extract containing about 0.3 mole of phenolic hydroxyl by detection, the ratio of moles number of nitrogen atoms in alkaloids to moles number of phenolic hydroxyl is 2.2/1. Seal the reaction kettle, turn on the heating, set a continuous stirring speed at 300 rps, stir and heat at a constant temperature of 110° C. for 30 minutes, and obtain a reaction compound of combined state “alkaloid-phenol” of (nicotine-arecoline extract)-(tea polyphenol-lily polyphenol).

According to the conventional preparation processes and equipment of HNB fuming colloid, a certain amount of the above-mentioned reaction compound is mixed into the fuming colloid to obtain a solid atomizing cartridge, and using the matching rod to obtain a HNB electronic atomizer containing the reaction compound of combined state “alkaloid-phenol” of (nicotine-arecoline extract)-(tea polyphenol-lily polyphenol), which is marked as “G2 atomizer”.

### Suction Test Results:

There are a total of 14 electronic atomizer samples in the above-mentioned 7 groups of Comparative Examples and Embodiments, using the same model of the atomizer in each group, and organize 20 person-time blind test of suction experience comparisons for each sample. The result table of comprehensive evaluation is as follows:

| | state of | | | primitive | | | | | aroma | |
|---|---|---|---|---|---|---|---|---|---|---|
| | alkaloid and | | | irritancy | evenness | appropriate | | | not | |
| | phenol in | | | of | of | throat | smoothly | fast | from | sour |
| Projects | e-liquid | e-liquid | atomizer | alkaloid | aerosol | sensation | silky | satisfaction | essence | smell |
| Comparative | free state of nicotine | A1 | A1 | acute | mixed | irritated | rough | slow | no | no |
| Example 1 | and cannabidiol | e-liquid | atomizer | | unevenuess | | | | | |
| Embodiment | reaction composition | A2 | A2 | no | evenly | comfortable | silky | fast | yes | no |
| 1 | of combined state | e-liquid | atomizer | | balanced | | | | | |
| | nicotine-cannabidiol | | | | | | | | | |
| Comparative | free state of nicotine, | B1 | B1 | acute | mixed | very | rough | slowy | no | no |
| Example 2 | arecoline, | e-liquid | atomizer | | unevenness | irritated | | | | |
| | cannabinoids | | | | | | | | | |
| Embodiment | reaction composition | B2 | B2 | no | evenly | comfortable | silky | fast | yes | no |
| 2 | of combined state | e-liquid | atomizer | | balanced | | | | | |
| | (nicotine + arecoline)- | | | | | | | | | |
| | cannabinolds | | | | | | | | | |
| Comparative | free state of | C1 | C1 | weak | mixed | weak | rough | slow | no | no |
| Example 3 | theophylline, | e-liquid | atomizer | | unevenness | | | | | |
| | carvacrol, | | | | | | | | | |
| | chrysophanol | | | | | | | | | |
| Embodiment | reaction composition | C2 | C2 | no | evenly | yes | silky | fast | yes | no |
| 3 | of combined state | e-liquid | atomizer | | balanced | | | | | |
| | theophylline- | | | | | | | | | |
| | (carvacrol + | | | | | | | | | |
| | chrysophanol) | | | | | | | | | |
| Comparative | free state of nicotine, | D1 | D1 | acute | mixed | very irritated | rough | slow | no | no |
| Example 4 | hygrine, phenol, | e-liquid | atomizer | | unevenness | | | | | |
| | sesamol | | | | | | | | | |
| Embodiment | reaction composition | D2 | D2 | weak | evenly | slightly | silky | fast | yes | no |
| 4 | of combined state | e-liquid | atomizer | | balanced | comfortable | | | | |
| | (nicotine + hygrine)- | | | | | | | | | |
| | (phenol + sesamol) | | | | | | | | | |
| Comparative | free state of | E1 | E1 | acute | mixed | very | rough | slow | no | no |
| Example 5 | arecoline, caffeine, | e-liquid | atomizer | | uneveness | irritated | | | | |
| | theobromine, | | | | | | | | | |
| | cannabidiol,eugenol, | | | | | | | | | |
| | carvacrol, catechol | | | | | | | | | |
| Embodiment | reaction composition | E2 | E2 | weak | evenly | slightly | silky | fast | yes | no |
| 5 | of combined state | e-liquid | atomizer | | balanced | comfortable | | | | |
| | (arecoline + caffeine + | | | | | | | | | |
| | theobromine) | | | | | | | | | |
| | (cannabidiol + eugenol + | | | | | | | | | |
| | carvacrol + | | | | | | | | | |
| | catechol) | | | | | | | | | |
| Comparative | free state of benzoic | F1 | F1 | no | evenly | slightly | slightly | little fast | no | yes |
| Example 6 | acid nicotine salt, | e-liquid | atomizer | | | comfortable | silky | | | |
| | cannabis polyphenol | | | | | | | | | |
| Embodiment | reaction composition | F2 | F2 | no | evenly | comfortable | silky | fast | yes | no |
| 6 | of combined state | e-liquid | atomizer | | balanced | | | | | |
| | nicotine-cannabis | | | | | | | | | |
| | polyphenol | | | | | | | | | |
| Comparative | free state of oxalate | / | G1 | weak | slightly | not | slightly | little fast | no | acute |
| Example 7 | nicotine salt, | | atomizer | | evenly | comfortable | silky | | | |
| | arecoline extract, | | | | | | | | | |
| | tea polyphenol, lily | | | | | | | | | |
| | polyphenol | | | | | | | | | |
| Embodiment | reaction composition | / | G2 | no | evenly | comfortable | silky | more fast | acute | no |
| 7 | of combined state | | atomizer | | balanced | | | | | |
| | (nicotine-arecoline | | | | | | | | | |
| | extract)- | | | | | | | | | |
| | (tea polyphenol | | | | | | | | | |
| | lily polyphenol) | | | | | | | | | |

According to the result table: the seven Embodiments are significantly better than the seven Comparative Examples respectively under the same conditions of the suction experience comparisons, indicating that the e-liquids in the Embodiments containing the reaction compound of alkaloid-phenol help to eliminate the original irritation of alkaloid, has no sour smell and can balance the aerosol, and increases the aroma not from essence. The analysis is: although the types of raw materials contained and the ratio of the molar number of alkaloid nitrogen to the molar number of phenolic hydroxyl are almost the same in the Comparative Examples and the Embodiments, the Comparative Examples simply mix those raw materials at room temperature, wherein the contained alkaloids and phenols are in free state each other in the e-liquid as well as in the atomizing cartridge, while in the Embodiments, the reaction compounds of alkaloids and phenols are formed by chemical reaction under heating and stirring in advance, and the e-liquids and atomizing cartridges contain the combined state “alkaloid-phenol”.

The ratio of moles number of nitrogen atoms in alkaloids to moles number of phenolic hydroxyl is (0.0001-10000)/1, and the reaction temperature is 40° C.-300° C. and continuously stirring for 0.1-6000 minutes to obtain the chemical reaction compound containing a combined state “alkaloid-phenol”; the e-liquid containing the reaction compound of “alkaloid-phenol” is better than the e-liquid which is simply mixed by directly adding phenol to the mixture of main solvent, essence, nicotine and organic acid nicotine salt at room temperature, and the suction experience is better. Theoretical analysis is as follows: in the Embodiments, a combined state “alkaloid-phenol” in the reaction compound of alkaloid and phenol is dispersed into the atomized aerosol, while in the Comparative Examples, the alkaloid and phenol are independent of each other to be atomized early or late due to the difference of their boiling points, causing the strong original irritation of alkaloids in aerosols, throat choking, uneven and rough mixing of aerosols, etc.

Comparing the e-liquids containing only free state alkaloid and phenol of Comparative Example 1-5 and the e-liquids prepared from nicotine salt of Comparative Example 6-7, the former e-liquids have no sour smell of nicotine salt, but because the e-liquid of Comparative Examples 1-5 do not promote chemical reaction to form the combined state “alkaloid-phenol”, and there are still a mixed uneven, rough and throat-choking suction experience during using.

To the surprise of the inventor, the e-liquid containing reaction compound of “alkaloid-phenol” of multiple alkaloids and multiple phenols is more effective than the e-liquid containing reaction compound of “alkaloid-phenol” of a single alkaloid and a single phenol, which is more likely to make the user feel satisfied quickly, comparing of Embodiment 6 and Embodiment 7. It is theoretically speculated that: the synergistic effect of reaction compound of “alkaloid-phenol” of various alkaloids and various phenol is easier to break through the blood-brain barrier and improve the transmission efficiency of alkaloids. The specific physiological mechanism of the human brain is currently unknown.

### Supplementary Experiment Content

The equipment and the detection method are very mature for the quantitative detection of the nitrogen atom in the alkaloid raw material and the molar number of the phenolic hydroxyl in the phenolic raw material, and all work can be implemented by those skilled in the art without creative work. The detection method is briefly described as follows:

Raw materials of alkaloids include but are not limited to the extracts from animal or plant containing alkaloids, purified alkaloids, and artificially synthesized alkaloids. The quantitative detection of nitrogen atoms in alkaloid raw materials is preferably performed using an elemental analyzer. The detection process is: the elemental analyzer is in the CHNS measurement mode, after weighing the sample, it enters a combustion tube and burns completely in a pure oxygen atmosphere, and the combustion products form CO₂, H₂O, N₂ and nitrogen oxides by a specific reagent, and then the gas enters the reduction tube to remove excess oxygen and reduce nitrogen oxides to N₂, and then realize gas separation through the purge and trap adsorption column or gas chromatography column, and then enter the thermal conductivity detector for quantitative detection, and finally calculate the nitrogen atoms content in the alkaloid raw material.

Furtherly, the moles number of nitrogen atoms in alkaloid raw materials can also be detected by chemical methods, spectroscopic methods, energy spectroscopic methods, etc. Nitrogen atoms in single alkaloids, multiple alkaloids, alkaloid extracts and alkaloid compositions are all suitable for the above quantitative detection methods.

Phenolic raw materials include but not limited to extracts from animal or plant containing phenols, purified phenols, and artificially synthesized phenols. The quantitative detection methods for phenolic hydroxyl in phenolic raw materials such as: colorimetric method, wherein excess ferrous tartrate reacts with phenolic hydroxyl groups of phenolic raw material to generate a purple-brown complex, the color of the solution is proportional to the number of phenolic hydroxyl; Folin's phenol method to determine the total phenol content, wherein, phosphotungstomolybdic acid can quantitatively oxidize phenolic compounds in alkaline solution, and W⁶⁺ can be reduced to W⁵⁺ blue color compounds, the degree of blue is proportional to the number of phenolic hydroxyl;

Furtherly, the moles number of phenolic hydroxyl in phenolic raw materials can also be quantitatively detected by gas chromatography, liquid chromatography and spectrophotometry. The above quantitative detection methods are all suitable for phenolic hydroxyl of single phenol, polyphenol, phenolic extracts and phenolic compositions.

To sum up, the reaction compound of alkaloid and phenol, e-liquid, atomization cartridge and electronic atomizer pointed out by the present invention can better the original alkaloid throat irritating taste through the reaction compound of alkaloid and phenol, which is beneficial for the mouth inhalation, and will improves the suction experience by without troubles such as “acidosis”, “acid excess” and “sour smell”, etc.

The above descriptions are only several Embodiments of the present invention, and are not intended to limit the patent scope of the present invention. Any equivalent structure or equivalent process transformation made by using the content of the description of the present invention, or directly or indirectly used in other related technical fields, are equally included within the scope of patent protection of the present invention.

What is claimed is:

1. An e-liquid, wherein the e-liquid comprises a reaction compound of alkaloid and phenol, and the production method of the reaction compound of alkaloid and phenol comprises the following steps:
add reactant alkaloid and reactant phenol in a reaction vessel, carry out heating;
heat and stir continuously for 0.1-6000 minutes at a temperature of 40° C. to 300° C., and then the reaction compound of alkaloid and phenol is prepared.

2. The e-liquid according to claim 1, wherein the alkaloid is a combination of at least one of betaine, theophylline, capsaicin, arecoline, caffeine, piperine, pepperine, nicotine, theobromine, leonurine, hygrine, red cousidine, sophora alkaloid, integerrimine, monocrotaline, lobeline, lobelialine, expectorantine, sanguinarine, maytansine, small berberine, berberine, tetrandrine, African tetrandrine, sanguinarine citrate, reserpine, pilocarpine, atropine, hyoscyamine, scopolamine, halosine, solanine, belladonna alkaline, fritillary base, aconitine, delphinium, galanthaminc, tripteryline, venomine, anabasine, coptisine, North American berberine, sinomenine, magnolialine, erpentine, n-methylcytisine, anagerline, bapufoline, matrine, oxymatrine, hydroxymatrine, dehydrogenation matrine, ginseng alkaloid, evodia alkaloid, schiff base, vinblastine, vincristine, cinchonaline, strychnine, rhynchophylline, isatidine, macaine, moringa alkaloid, kudzu root carnitine, guarana alkaloid, ganoderma alkaloid, L-carnitine, lotus leaf alkaloid, cactus alkaloid, seabuckthorn alkaloid, plum essence, papaya alkaloid, chive alkaloid, epimedium alkaloid, cynomex alkaloid, cistanche alkaloid, actinidinine, lycorine, aloe alkaloid, mulberry leaf carnitine, mulberry leaf alkaloid, ginger alkaloid, golden clover alkaloid, hippocampus choline, angelica, chuanbei alkaloid, ergot alkaloid, ergometrine, sophocarpine, haematine, dauricine, corydine aureus, genus alkaloid, silkworm excrement alkaloid, diconazole alkaloid, emetine, qingfengteng alkaloid, chuanxiong alkaloid, oat alkaloid, ryegrass alkaloid, celandine total alkaloids, dandelion alkaloid, corn silk alkaloid, cocklebur alkaloid, jatrorrhizine, hibiscus alkaloid, camptothecin, phellodendron, quinoa alkaloid, and the phenol is a combination of at least one of catechol, tea polyphenol, maltol, ethyl maltol, paconol, phenol, cannabinol, cannabidiol, cannabigerol, cannabichromene, alkylphenol, acetaminophen, nitrophenol, naphthol, chlorophenol, biquinone, resorcinol, hydroquinone, pyrogallol, phloroglucinol, pyroglucinol, cresol, tocopherol, tocotrienol, proanthocyanidin polyphenol, honey polyphenol, olive polyphenol, chocolate polyphenol, pine polyphenol, eugenol, isocugenol, rhubarb phenol, Ginkgo biloba polyphenol, algae polyphenol, seaweed polyphenol, brown algae polyphenol, raspberry polyphenol, santalol, muskol, thymol, agarol, star anise polyphenol, eucalyptus phenol, oregano phenol, carvacrol, zingerol, gossypol, agrimonol, bakuchiol, ligninol, cardanol, geranol, schisandrol, cholesterol, chlorine myol, 4-aminophenol, orcinol, clomiphene, folinol, phenylselenol, dendrobium phenol, kaempferol, taxol, honokiol, total magnolol, sesamol, apple polyphenol, peach polyphenol, pear polyphenol, grapefruit polyphenol, bayberry polyphenol, citrus polyphenol, macaque peach polyphenol, banana polyphenol, orange polyphenol, papaya polyphenol, cantaloupe polyphenol, chestnut shell polyphenol, chestnut bract polyphenol, loquat polyphenol, cocoa polyphenol, cocoa bean polyphenol, monk fruit polyphenol, lotus seed polyphenol, potato polyphenol, jujube polyphenol, sandy date polyphenol, garlic polyphenol, onion polyphenol, corn polyphenol, yam polyphenol, sorghum polyphenol, peanut kernel polyphenol, peanut red coat polyphenol, grape polyphenol, red wine polyphenol, mung bean polyphenol, cowpea polyphenol, wolfberry polyphenol, sunflower seed polyphenol, lily polyphenol, sealwort polyphenol, polygonatum polyphenol, camellia oleifera polyphenol, fungus polyphenol, lotus root polyphenol, pueraria polyphenol, fennel polyphenol, thistle polyphenol, evening primrose polyphenol, coriander polyphenol, cumin polyphenol, lavender polyphenol, rose polyphenol, chrysanthemum polyphenol, jasmine polyphenol, sophora polyphenol, osmanthus polyphenol, gardenia polyphenol, plum polyphenol, guaiacol, 4-methylguaiacol, 2-isopropylphenol, 2-methoxy-4-vinylphenol, dimethoxyphenol, and 3,4-dimethylphenol.

3. The e-liquid according to claim 1, wherein the e-liquid further comprises solvent and essence, the weight ratio of the reaction compound of alkaloid and phenol in the e-liquid is 0.001% to 96%, and the weight ratio of the solvent in the e-liquid is 3% to 98.999%, and the weight ratio of the essence in the e-liquid is 1% to 30%, the production process sequence of the e-liquid is: first, prepare the reaction compound of alkaloid and phenol in a reaction vessel, and then mix the prepared reaction compound of alkaloid and phenol with solvent and essence to form the e-liquid.

4. The e-liquid according to claim 3, wherein the solvent is a combination of at least one of propylene glycol, glycerin, water, butylene glycol, ethanol, and medium-chain triglyceride, and the essence is a combination of at least one of sweetener, cooling agent, fruit essence, tobacco essence, meat essence.

5. An atomizing cartridge, wherein the atomizing cartridge is a liquid atomizing cartridge containing the e-liquid as claimed in claim 1, or the atomizing cartridge is a solid atomizing cartridge made from the e-liquid as claimed in claim 1.

6. An electronic atomizer, wherein the electronic atomizer contains the atomizing cartridge as claimed in claim 5, and the electronic atomizer is at least one of a vapor electronic atomizer that consumes liquid atomizing cartridge and a HNB electronic atomizer that consumes solid atomizing cartridge.
